(19) United States
(12) Patent Application Publication
(10) Pub. No.: US 2019/0254855 A1
(43) Pub. Date: Aug. 22, 2019

(54) Device and Method for Enhancing and Facilitating Correct Spinal Alignment

(71) Applicant: AdBack, LLC, Los Angeles, CA (US)

(72) Inventors: Wayne Daniels, Los Angeles, CA (US); Alii R. Ontiki, Los Angeles, CA (US); George W. Cranford, Los Angeles, CA (US)

(21) Appl. No.: 15/563,610

(22) PCT Filed: Apr. 4, 2016
(86) PCT No.: PCT/US16/25941

§ 371 Date: Oct. 9, 2018

Related U.S. Application Data
(60) Provisional application No. 62/143,010, filed on Apr. 3, 2015.

(51) Int. Cl. A61F 5/02

(52) U.S. Cl. CPC A61F 5/026; A61F 5/024

(57) ABSTRACT

A device for enhancing and facilitating correct spinal alignment comprising a flat or rounded bar and a series of removable pads, contoured to rest below the base of the skull, i.e., at the first vertebra of the cervical spine (or atlas vertebra), with the lower end of the bar placed at the lumbar region of a user. The dimensions of the pad in use are determined by the degree of cervical alignment required and are adjustable. The device may be worn on the body held in place with straps, affixed in clothing, backpacks, vests, or any other wearable garment or accessory, or attached to a bracket that may be attached or affixed to another object, such as a car seat, chair, etc. In use, the device encourages simultaneous physical contact of the user's atlas vertebra, thoracic spine, and lower lumbar spine with the flat or rounded bar.

### CROSS-REFERENCE To RELATED APPLICATION

This application claims the priority to U.S. Provisional Patent Application Ser. No. 62/143,010, entitled “Device and Method for Enhancing and Facilitating Correct Spinal Alignment,” filed on Jun. 3, 2015, to inventors Wayne Daniels, Alii R. Ontiki, and George W. Cranford IV, the disclosure of which is incorporated by reference herein in its entirety.

### BACKGROUND OF THE INVENTION

### 1. Field of the Invention

The invention relates generally to devices and methods that enhance a person's health and well-being, and more particularly, to devices and methods that operate to improve and properly maintain a person's posture.

### 2. Description of the Related Art

Today, many individuals have poor posture which may adversely affect their physical and mental health. For example, poor posture is generally unattractive. Many people perceive individuals with upright posture as interested and alert, while individuals who slouch or stoop may be viewed as lazy, awkward, or frail. These perceptions influence whether others positively or negatively view a person's work or mental condition. In addition to being socially undesirable, poor posture can cause various health problems. With continual poor posture, these health problems can include curvature of the spine and a chronic stooping condition.

Poor posture is posture that results from certain muscles tightening up or shortening while others lengthen and become weak, which often occurs as a result of one's daily activities. The different factors that can impact a person's posture include occupational activities as well as biomechanical factors such as force and repetition. In general, computer use is problematic concerning posture because a person using a computer, either at work or for personal pleasure, may sit at a computer without proper posture for extended periods of time, that is, slouched and hunched over, peering at a computer monitor, perhaps while sitting cross-legged or with legs curled under seats.

Other sources of poor posture are repetitive motion without frequent breaks and also sitting with poor posture for long periods without interruption. If one spends a substantial part of one's day in a certain position without frequent reprieves, the spine tends to orient itself to that position. For example, if someone is constantly leaning over to pick up objects, gradually the spine will start to develop a more exaggerated forward curve of the thoracic spine. In other words, poor posture may be caused either by prolonged periods of repeated motions, or by remaining fixed in one particular position for prolonged periods.

From the foregoing it is evident that poor posture is at present a common affliction suffered by many people and thus there is a need for a device and methods of using the device that will enable a person to improve his or her posture. Such a device should be able to be worn by the user for extended periods of time, while undertaking various activities throughout the day, and should be lightweight, comfortable and adjustable to the user's changing posture.

### SUMMARY

Devices and methods of correcting, improving, and properly maintaining a person's posture and spinal alignment are disclosed. Such a device in accordance with the invention may include a flat or rounded metal or plastic Atlas bar and a removable, adjustable Atlas pad affixed to a top end of the Atlas bar, where the Atlas pad is contoured to rest below the base of the skull, i.e., at the first vertebra of the cervical region of the spine (or Atlas vertebra), of a user. Being removable, Atlas pads of varying dimensions may be changed as required to accommodate a user's particular need. Additionally, an Atlas bar may be configured to be adjustable relative to the Atlas bar. The bottom end of the Atlas bar rests upon the lumbar region of the user, where this bottom end may also include a pad or padding to make the device more comfortable to the user.

The Atlas bar may be worn on the body and held in place by two or more straps, which may take the form of a vest or corset comprised of lightweight nylon webbing or other like materials, and worn on the body in the form of clothing with quick-release fasteners. The webbing or the like may also be attached by way of brackets to backpacks, office desks, car seats, etc., or other stationary objects.

In use, the Atlas bar encourages simultaneous physical contact of the Atlas vertebra, the thoracic region of the spine, and lower lumbar region of the spine of the user. When used in various settings, the device facilitates correct spinal posture, creates proprioceptive awareness and recruitment of suboccipital muscles, and increases spinal tension and adaptability in movement. As the user's spinal posture improves, the dimensions of the Atlas pad may change. That is, the distance between the center of the arc where the Atlas pad rests on the Atlas vertebra and the Atlas bar itself decreases as the posture improves, as the misalignment of the spine decreases.

The device is intended for use in all types of settings and for extended periods of time. For example, the device may be used while working in an office or at a computer for several hours, working in an occupation that requires physical movement and labor, or while exercising or participating in sporting activities.

Other systems, methods, features and advantages of the invention will be or will become apparent to one with skill in the art upon examination of the following figures and detailed description. It is intended that all such additional systems, methods, features and advantages be included within this description, be within the scope of the invention, and be protected by the accompanying claims.

### BRIEF DESCRIPTION OF THE FIGURES

The examples of the invention described below can be better understood with reference to the following figures. The components in the figures are not necessarily to scale, emphasis instead being placed upon illustrating the principles of the invention. In the figures, like reference numerals designate corresponding parts throughout the different views.

FIG. 1 shows a perspective view of an example implementation of an assembled Atlas bar device in accordance with the invention.

FIG. 2A shows a perspective view of the Atlas bar sheath of the assembled Atlas bar device of FIG. 1.

FIG. 2B shows a perspective view of the Atlas bar of the assembled Atlas bar device of FIG. 1.

FIG. 2C shows a perspective view of the Atlas pad of the assembled Atlas bar device of FIG. 1.

FIG. 2D shows a perspective view of the Atlas pad holder of the assembled Atlas bar device of FIG. 1.

FIG. 2E shows a perspective view of the bottom Atlas bar holder of the assembled Atlas bar device of FIG. 1.

FIG. 3A shows a top plan view of an example implementation of an Atlas pad in accordance with the invention.

FIG. 3B shows a top plan view of an Atlas pad similar to that shown in FIG. 3A but with different dimensions.

FIG. 3C shows a top plan view of another Atlas pad similar to that shown in FIG. 3A but with different dimensions.

FIG. 4A shows a top plan view of a sketch of another example implementation of an Atlas pad in accordance with the invention that illustrates one dimension of an Atlas pad.

FIG. 4B shows a top plan view of a sketch of the Atlas pad shown in FIG. 4A that illustrates a changed dimension of an Atlas pad.

FIG. 4C shows a top plan view of a sketch of the Atlas pad shown in FIG. 4A that illustrates another changed dimension of an Atlas pad.

FIG. 4D shows a top plan view of a sketch of the Atlas pad shown in FIG. 4A that illustrates yet another changed dimension of an Atlas pad.

FIG. 5 shows an exploded perspective view of yet another example implementation of an Atlas pad holder and an Atlas pad in accordance with the present invention.

FIG. 6 shows a perspective view of the Atlas pad holder and the Atlas pad of FIG. 5 placed against the Atlas vertebrae of a user.

FIG. 7A shows a front perspective view of an example implementation of an assembled Atlas bar device in accordance with the invention together with an example of an embodiment of straps that may be used to attach the assembled Atlas bar device to a user.

FIG. 7B shows a rear perspective view of the assembled Atlas bar device and embodiment of straps shown in FIG. 7A.

FIG. 8A shows a front elevational view of another example implementation of an assembled Atlas bar device in accordance with the invention together with another example of an embodiment of straps that may be used to attach the assembled Atlas bar device to a user.

FIG. 8B shows a rear elevational view of the assembled Atlas bar device and embodiment of straps shown in FIG. 8A.

FIG. 8C shows a front perspective view of the assembled Atlas bar device and embodiment of straps shown in FIG. 8A.

FIG. 8D shows a rear perspective view of the assembled Atlas bar device and embodiment of straps shown in FIG. 8A.

FIG. 8E shows a top plan view of the assembled Atlas bar device and embodiment of straps shown in FIG. 8A.

FIG. 9 shows a front elevational view of yet another example implementation of an Atlas bar device in accordance with the invention together with another example of an embodiment of straps that may be used to attach the assembled Atlas bar device to a user.

### DETAILED DESCRIPTION

In the following description of the preferred and various alternative embodiments, reference is made to the accompanying drawings that form a part hereof, and in which is shown by way of illustration a specific embodiment in which the invention may be practiced. It is to be understood that other embodiments may be utilized and various structural changes may be made without departing from the spirit and scope of this invention.

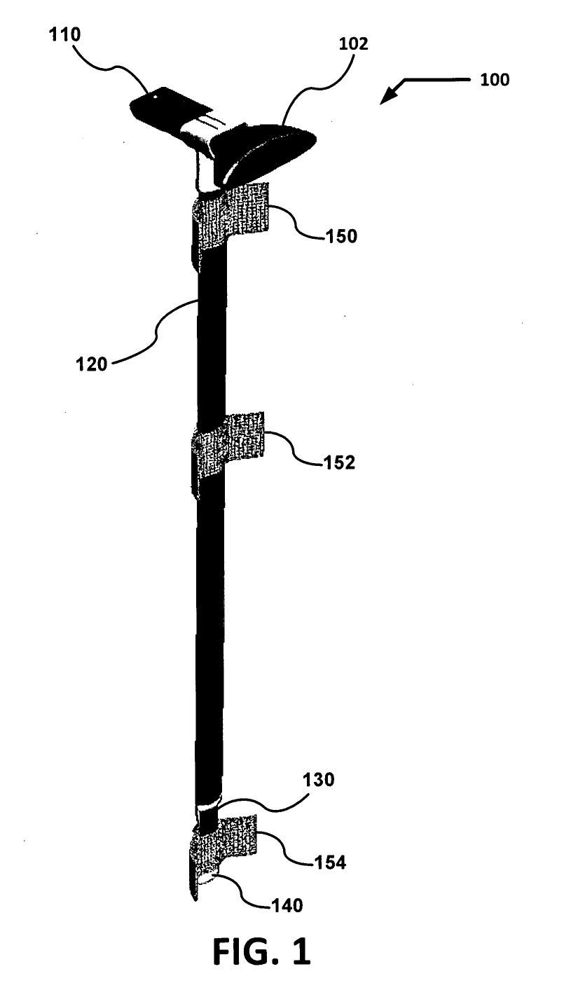

FIG. 1 shows a perspective view of an example implementation of an assembled Atlas bar device 100 in accordance with the invention. In FIG. 1, an Atlas pad 102 is shown inserted into an Atlas pad holder 110. The Atlas pad holder 110 is then placed onto the top of Atlas bar sheath 120. Inside the Atlas bar sheath 120, the Atlas bar 130 is movably inserted such that the Atlas bar 130 may be adjusted to the height of the user. At the bottom of the Atlas bar 130, a bottom Atlas bar holder 140 may be placed over the end of the Atlas bar 130, such that the bottom Atlas bar holder 140 may contact the lower lumbar region of the spine of the user.

Also shown in FIG. 1 are portions of straps or webbing referred to as an upper strap 150, a middle strap 152, and a lower strap 154. These straps are attached to either the Atlas bar sheath 120 or the Atlas bar 130, and may be part of a vest or corset-type of clothing (not shown) that may be worn by a user of the assembled Atlas bar device 100.

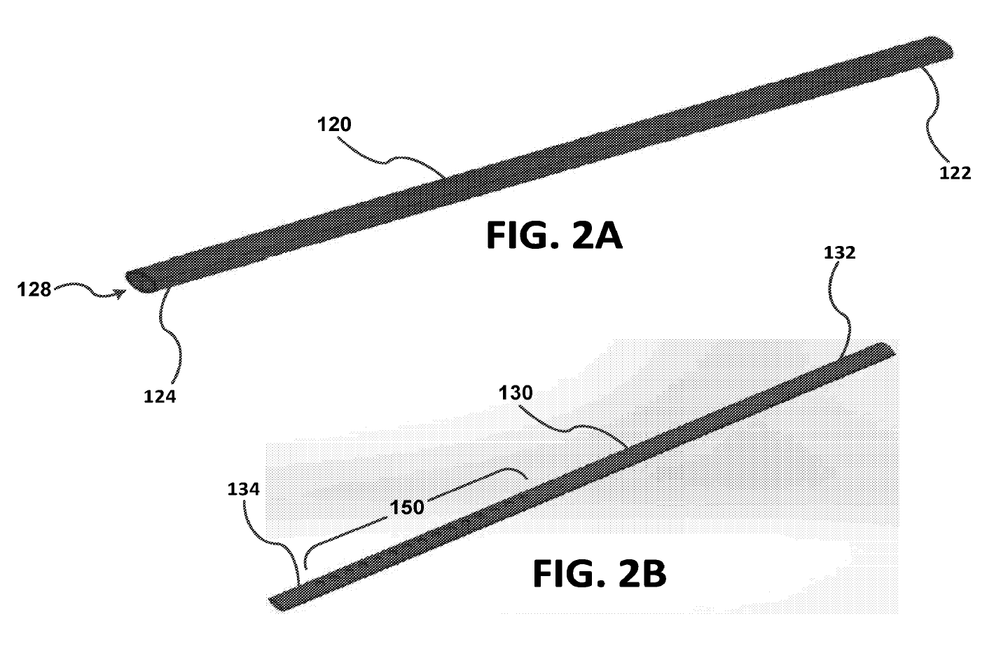

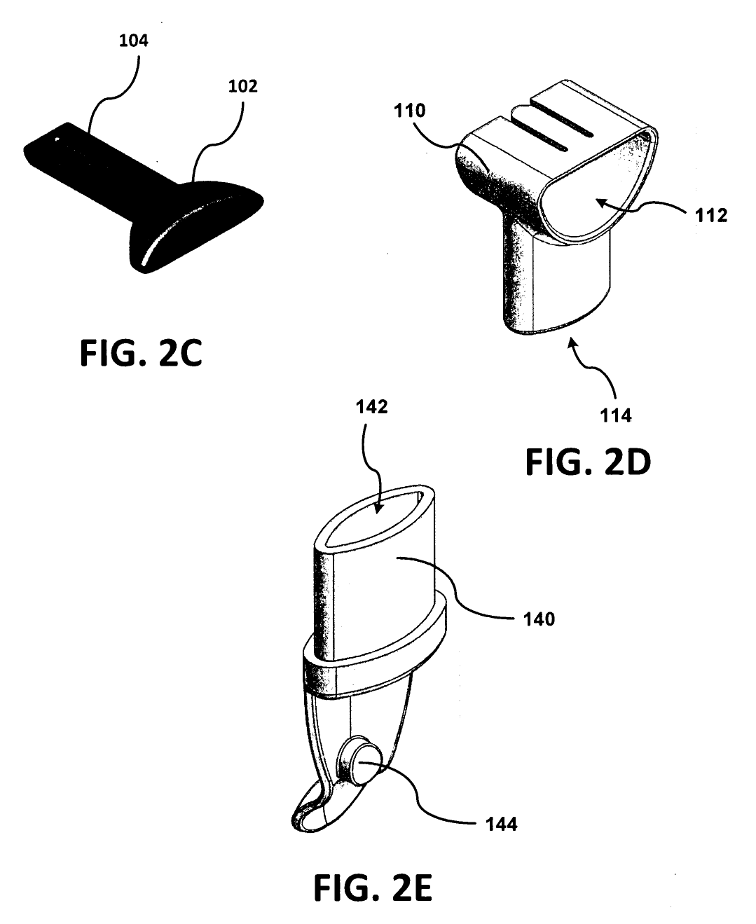

Turning to FIG. 2A, a perspective view of the Atlas bar sheath 120 is shown, with the Atlas bar sheath 120 having a top end 122 and a bottom end 124. The Atlas bar sheath 120 also has a cylindrical cavity 128 running along its entire length, where the cylindrical cavity 128 is configured to receive the Atlas bar 130, a perspective view of which is shown in FIG. 2B. The Atlas bar 130 also has a top end 132 and a bottom end 134. The Atlas bar 130 may also include a plurality of holes 150, evenly spaced along the bottom of the Atlas bar 130, which holes together with push-button 144 (see FIG. 2A) allow the Atlas bar 130 to be adjustable within the Atlas bar sheath 120.

FIG. 2C shows a perspective view of the Atlas pad 102 of the assembled Atlas bar device 100 of FIG. 1. The Atlas pad 102 may include a flange 104, which adjustably fits into slot 112 of the Atlas pad holder 110 that is shown in FIG. 2D. The Atlas pad holder 110 may also include a cavity 114, which may be fitted over the top end 122 of the Atlas bar sheath 120.

FIG. 2E shows a perspective view of the bottom Atlas bar holder 140 of the assembled Atlas bar device 100 of FIG. 1. The bottom Atlas bar holder 140 may include a slot 142, through which the bottom end 134 of the Atlas bar 130 is adjustably inserted. Once inserted and adjusted to its desired position, the Atlas bar 130 may be held in place by a spring-loaded push-button 144.

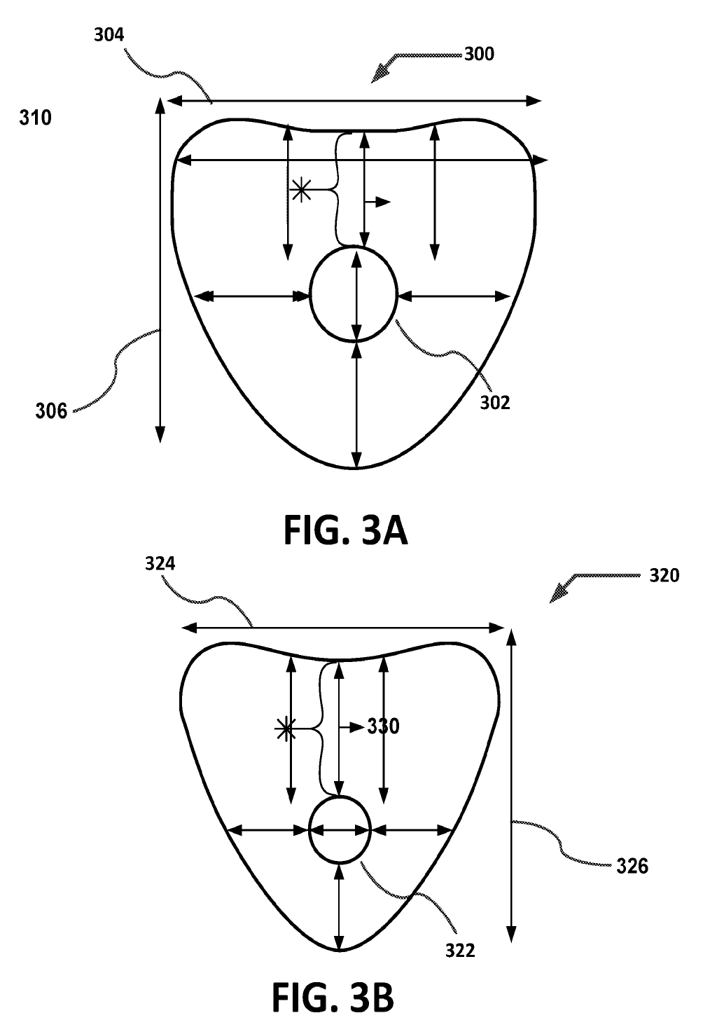

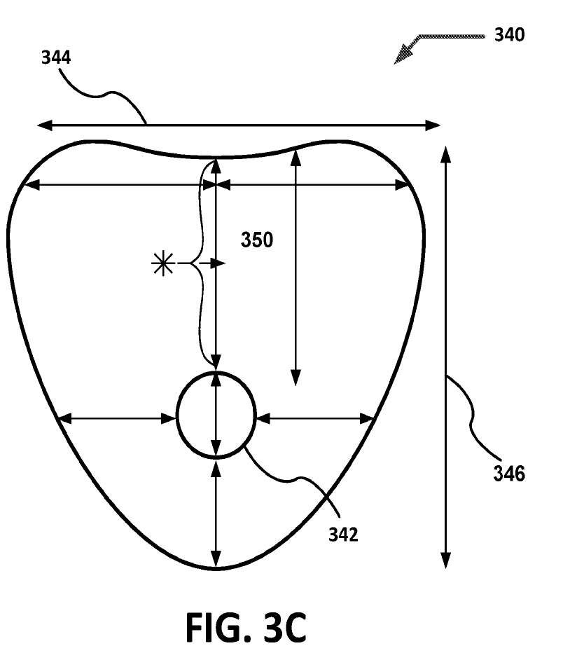

FIG. 3A shows a top plan view of another example implementation of an Atlas pad 300 in accordance with the invention. In this particular implementation, the Atlas pad 300 is configured to fit over a rounded Atlas bar (not shown) having a diameter of approximately ¾″, and accordingly, there is a hole 302 having the approximately the same diameter in Atlas pad 302.

The Atlas pad 300 has a width 304 of approximately 3 ½″ and a length 306 of approximately 3″. The most significant dimension of the Atlas pad 300 is the dimension 310, which is the distance between the center of the arc that touches and is contoured to rest at the Atlas vertebra of a user, and the point of contact with the Atlas bar. In Atlas pad 300, this dimension 310 is 1″.

Turning to FIGS. 3B and 3C, Atlas pads 320 and 340, respectively, are shown. The Atlas pad 320 has a width 324 of approximately 3 ½″ and a length 326 of approximately 3″. The Atlas pad 340 has a width 344 of approximately 4″ and a length 346 of approximately 3 ¾″. In Atlas pad 320, the dimension 330 is 1 ½″, and in Atlas pad 340, the dimension 350 is 2″. Accordingly, as noted above, Atlas pads 320 and 340 are each respectively used by a user with a greater degree of posture misalignment; as the distance between the center of the arc that touches the Atlas vertebra of a user, and the point of contact with the Atlas bar, increases in each embodiment. In general, this distance may be between and including 1″ to 6″.

As for the Atlas bar itself, it may be circular or rounded, elliptical, or flat. The Atlas bar may be made of metal, wood, plastic (i.e., ultra-high molecular weight polyethylene, polypropylene, polytetraflouroethylene, high-density polyethylene, and polyurethane), fiberglass, carbon fiber, foam (high-, medium-, or low-density, i.e., EVA/polyolefin, polyurethane), etc.

The Atlas pads may be made of metal, rubber, wood, foam (high-, medium-, or low-density, i.e., EVA/polyolefin), plastic (i.e., ultra-high molecular weight polyethylene, polypropylene, polytetraflouroethylene, high-density polyethylene, low-density polyethylene, and polyurethane), leather, etc., and the like.

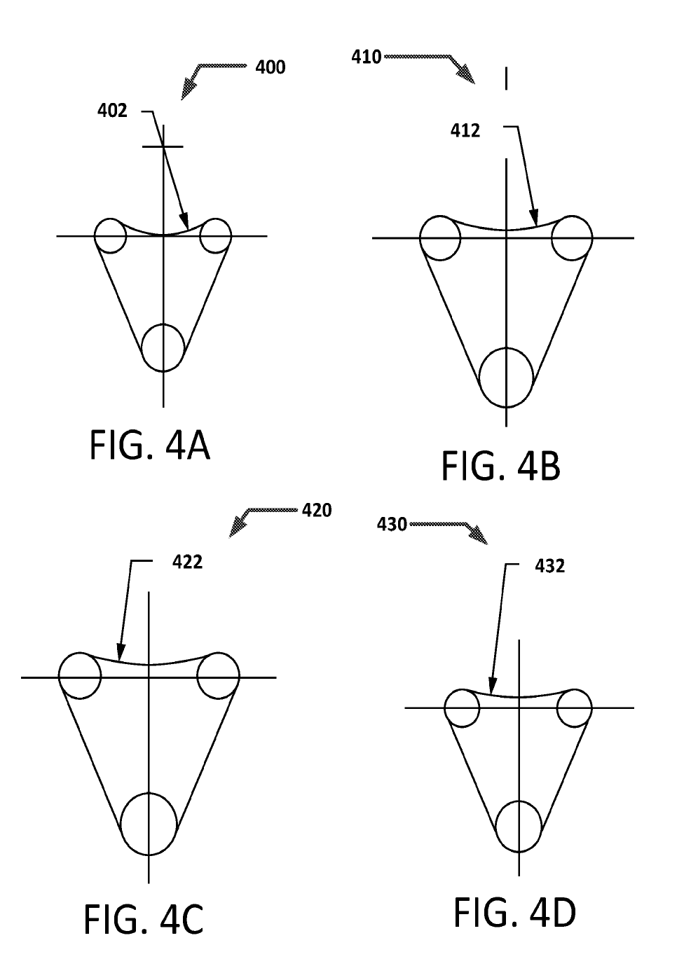

FIG. 4A shows a top plan view of a sketch of another example implementation of an Atlas pad 400 in accordance with the invention that illustrates one dimension of an Atlas pad. In FIG. 4A, radius 402 represents a radius of 2″, which defines the arc of the Atlas pad that touches and is contoured to rest at the Atlas vertebra of a user. Likewise, FIGS. 4B, 4C, and 4D show Atlas pads 410, 420, and 430, respectively, with radius 412 equal to 3″, radius 422 equal to 3″, and radius 432 equal to 5″, respectively. In general, as the radius increases, the depth of the arc decreases.

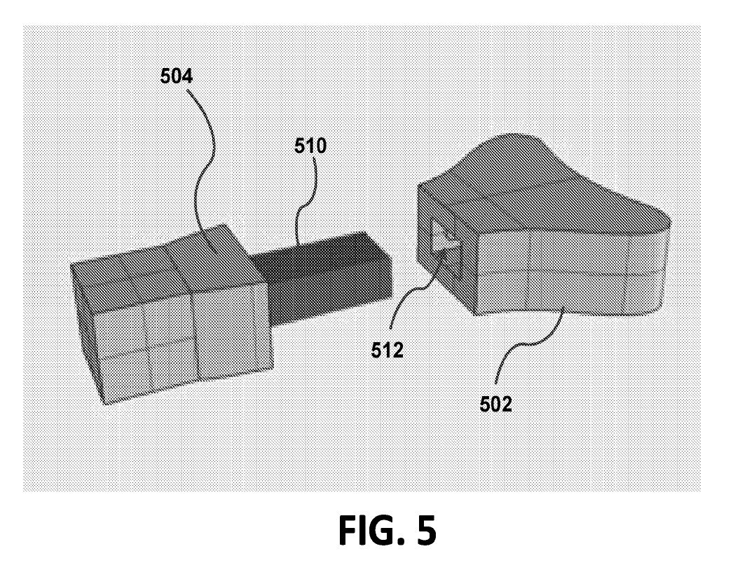

Turning to FIG. 5, yet another example implementation of an Atlas pad holder and an Atlas pad in accordance with the present invention is shown. Atlas pad 502 is shown with a slot 512, which is configured to receive the flange 510 of the Atlas pad holder 504. When the flange 510 is inserted into the slot 512, the Atlas pad 502 may be held in place by a locking mechanism (not shown) in the Atlas pad 502. The Atlas pad holder 504 may then be attached to an Atlas bar sheath as shown in FIG. 1.

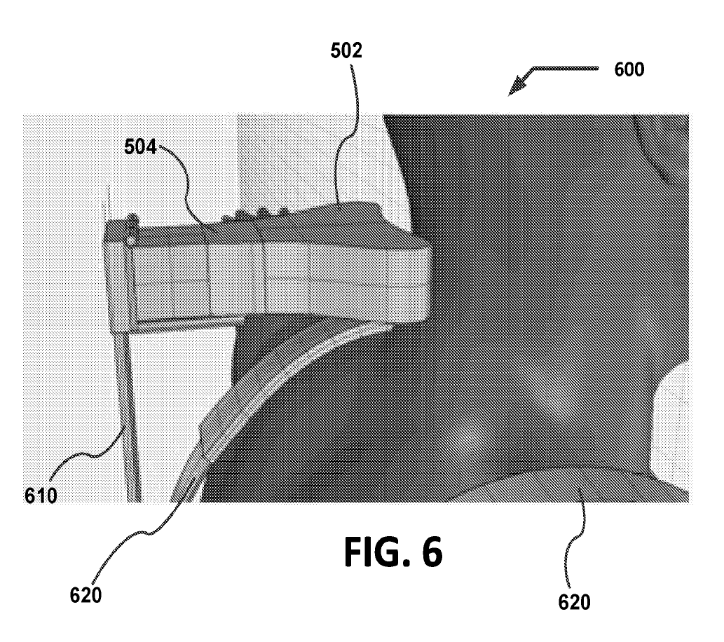

FIG. 6 shows a perspective view of the Atlas pad holder 504 and the Atlas pad 502 of FIG. 5 placed against the Atlas vertebrae of a user 600. In FIG. 6, the assembly of the Atlas pad holder 504 and the Atlas pad 502 is shown attached to an Atlas bar 610, which in turn is attached to the user by means of a vest comprising straps. Portions of the vest are shown as straps 620 in FIG. 6.

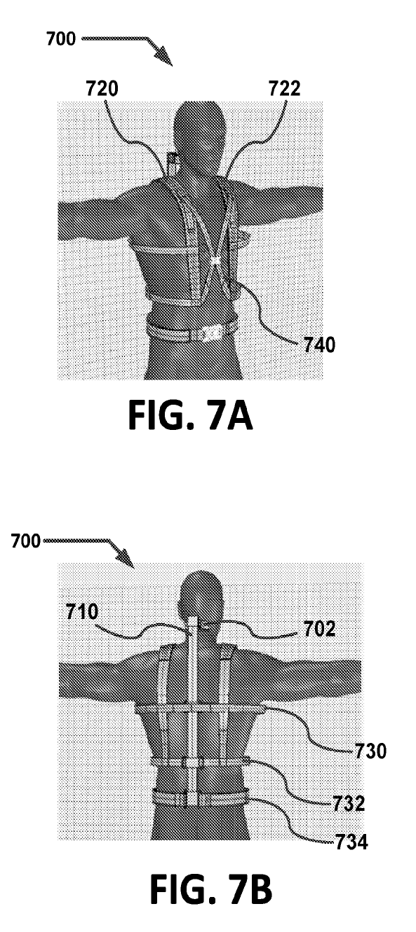

FIG. 7A shows a front perspective view of an example implementation of an assembled Atlas bar device 700 in accordance with the invention together with an example of an embodiment of straps that may be used to attach the assembled Atlas bar device to a user. In FIG. 7A, a right shoulder strap 720 and a left shoulder strap 722 are shown attached to an X-shaped brace 740, which supports the Atlas bar device 700 on the torso of a user.

Turning to FIG. 7B, which shows a rear perspective view of the assembled Atlas bar device 700 shown in FIG. 7A, Atlas pad 702 is shown attached to Atlas bar 710, with Atlas pad 702 shown in place at the Atlas vertebra of the user. Also shown are upper horizontal strap 730, middle horizontal strap 732, and lower horizontal strap 734, which are fitted around the torso of the user. Upper horizontal strap 730 and middle horizontal strap 732 are both attached to the right shoulder strap 720 and the left shoulder strap 722, while lower horizontal strap 734 fits around the waist of the user and includes a quick-fastener. All of these straps may include adjustment mechanisms (not shown), such as Velcro® fasteners, that allow the user to adjust each of the straps as needed.

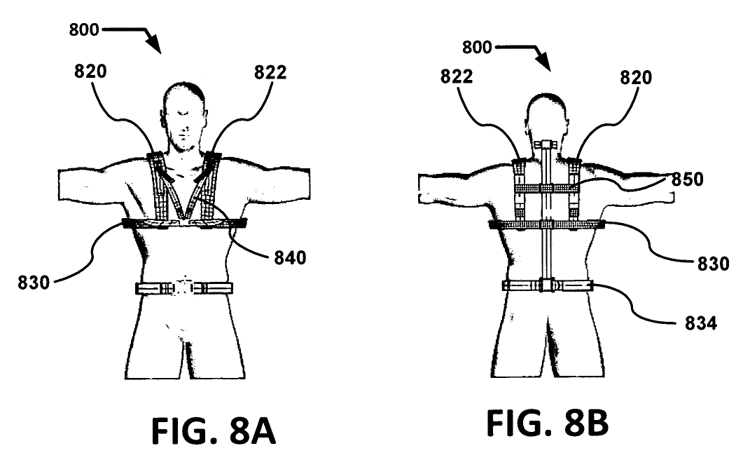

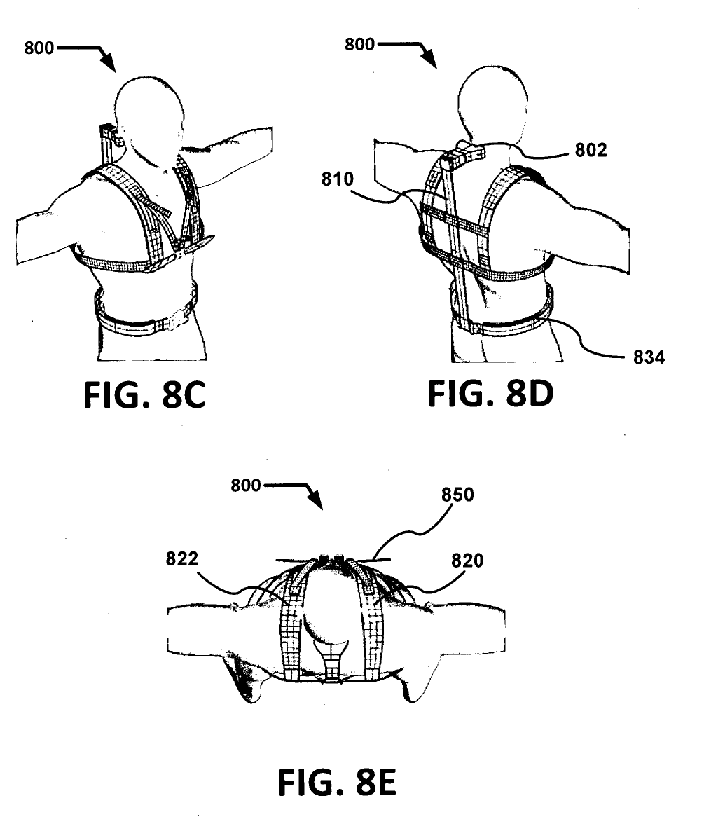

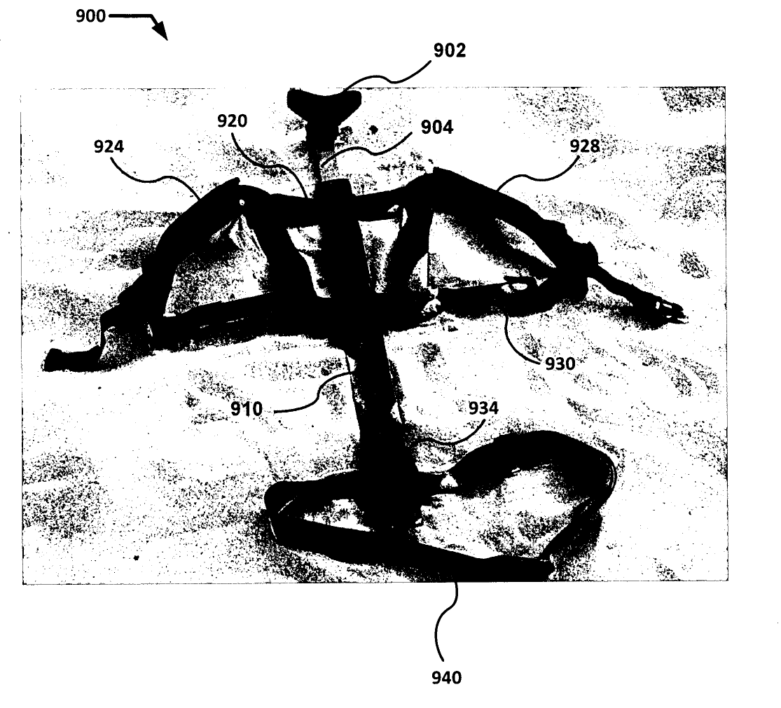

FIG. 8A shows a front elevational view of another example implementation of an assembled Atlas bar device in accordance with the invention, together with another example of an embodiment of straps that may be used to attach the assembled Atlas bar device to a user. In FIG. 8A, a right shoulder strap 820 and a left shoulder strap 822 are shown attached to a V-shaped brace 840, which is connected to upper horizontal strap 830 and which supports the Atlas bar device 800 on the torso of a user.

Turning to FIG. 8B, which shows a rear perspective view of the assembled Atlas bar device 800 shown in FIG. 8A, a cross brace 850 is shown attached to the right shoulder strap 820 and the left shoulder strap 822, and also to the upper horizontal strap 830. A lower horizontal strap 834, which fits around the waist of the user, is also shown.

FIG. 8C shows a front perspective view of the assembled Atlas bar device and embodiment of straps shown in FIG. 8A. Turning to FIG. 8D, which is a rear perspective view of the assembled Atlas bar device and embodiment of straps shown in FIG. 8A, Atlas pad 802 is shown attached to Atlas bar 810, with Atlas pad 802 shown in place at the Atlas vertebra of the user. Also shown is lower horizontal strap 834, which fits around the waist of the user and is attached to the bottom end of the Atlas bar 810. FIG. 8E shows a top plan view of the assembled Atlas bar device 800, where the right shoulder strap 820 and the left shoulder strap 822 are connected to adjustment means 850, which may be used to adjust the shoulder straps. These adjustment means may include Velcro® fasteners, side-release buckles, or fastener systems that allow the user to adjust each of the straps as needed.

FIG. 9 shows a front elevational view of yet another example implementation of an Atlas bar device 900 in accordance with the invention together with another example of an embodiment of straps that may be used to attach the assembled Atlas bar device to a user. The Atlas bar device 900 includes an Atlas pad 902 attached to Atlas bar 904, which is shown inserted into Atlas bar sheath 910. The Atlas bar sheath 910 is attached to the torso of the user by various straps, which may include a right shoulder strap 924 and a left shoulder strap 928, which are shown attached to a bracket-shaped strap 920. The bracket-shaped strap 920 is placed on the back of the user and held in place by a chest strap 930, which goes around the chest of the user.

Atlas bar device 900 may also include a lumbar pad 934, which fits into the lumbar region of the spine of the user and helps support and steady the Atlas bar device 900 on the torso of the user. Atlas bar device 900 may also include a waist strap 940, which also helps support the Atlas bar device 900 on the user. The chest strap 930 and the waist strap 940 both are shown with side-release buckles, but other types of fasteners, such as Velcro®, may be used.

The foregoing description of one or more implementations has been presented for purposes of illustration and description. It is not exhaustive and does not limit the claimed inventions to the precise form disclosed. Modifications and variations are possible in light of the above description or may be acquired from practicing the invention.

What is claimed is:

1. A device for enhancing and facilitating correct spinal alignment, the device comprising:
a flat or rounded Atlas bar configured to be placed in simultaneous physical contact with the Atlas vertebra, the thoracic region, and the lower lumbar region of the spine of a user, and having a top end and a bottom end;
an Atlas pad removably placed at the top end of the Atlas bar, contoured to rest at the Atlas vertebra of the user, and adjustable dependent on a degree of misalignment of the head and the spine of a user; and
an attachment means for affixing the Atlas bar and the Atlas pad to the back of the user.

2. The device for enhancing and facilitating correct spinal alignment of claim 1, wherein the attachment means comprises a plurality of shoulder straps and a plurality of horizontal straps.

3. The device for enhancing and facilitating correct spinal alignment of claim 2, wherein the shoulder straps and the horizontal straps are made from nylon or polypropylene webbing.

4. The device for enhancing and facilitating correct spinal alignment of claim 1, wherein the Atlas pad comprises:
a front surface contoured to conform to the Atlas vertebra of the user;
a distance between the front surface and the Atlas bar dependent on a degree of misalignment between the head and the spine of a user; and
attachment means for attaching the Atlas pad to the Atlas bar.

5. The device for enhancing and facilitating correct spinal alignment of claim 1, further comprising an Atlas bar sheath having a cylindrical cavity positioned along a length of the Atlas bar sheath and configured to receive and enclose the Atlas bar.

6. The device for enhancing and facilitating correct spinal alignment of claim 5, further comprising an Atlas pad holder having a cavity configured to fit securely over the top end of the Atlas bar.

7. The device for enhancing and facilitating correct spinal alignment of claim 6, where the Atlas pad further comprises a flange configured to securely fit into a slot of the Atlas pad holder.
